(19) United States
(12) Patent Application Publication
(10) Pub. No.: US 2022/0151582 A1
(43) Pub. Date: May 19, 2022

(54) SYSTEM AND METHOD FOR ASSESSING PULMONARY HEALTH

(71) Applicant: Acorai AB, Domsten (SE)

(72) Inventor: Filip Ludwig Peters, Domsten (SE)

(73) Assignee: Acorai AB, Domsten (SE)

(21) Appl. No.: 17/499,837

(22) Filed: Oct. 12, 2021

Related U.S. Application Data
(63) Continuation-in-part of application No. 17/099,772, filed on Nov. 16, 2020, now Pat. No. 11,232,866.

(51) Int. Cl. A61B 7/00; A61B 5/332; A61B 5/282; A61B 5/339; A61B 5/00; A61B 5/091; A61B 5/055; A61B 5/01

(52) U.S. Cl. CPC A61B 7/003; A61B 2560/0214; A61B 5/282; A61B 5/339; A61B 5/7267; A61B 5/4842; A61B 5/681; A61B 5/002; A61B 5/0035; A61B 5/091; A61B 5/055; A61B 5/01; A61B 2562/0219; A61B 2562/0271; A61B 5/332

(57) ABSTRACT

Disclosed are systems and methods for assessing pulmonary health. An example system includes a handheld electronic device (HED); a casing; and at least one circuit board. The HED includes a display screen, a processor, and a software application. The casing includes a plurality of ECG electrodes that are placed on the outer surface of the casing and at least one diaphragm. The ECG electrodes capture the electrophysiological data of the user. The circuit board is configured within the casing and electrically connected with the ECG electrodes and a microcontroller. The circuit board is further connected to at least one sound transducer and at least one Inertial Measurement Unit (IMU) sensor. The sound transducer captures pulmonary signals indicative of pulmonary health. The IMU sensor captures seismic and gyroscope signals indicative of the pulmonary health of the user and the orientation of the casing. The diaphragm enhances the pulmonary audio signals captured by the sound transducer. The microcontroller transmits the pulmonary health data to at least one of the HED and a computing device.

### CROSS-REFERENCE OF RELATED APPLICATION(S)

This application is a continuation-in-part of and claims priority to and benefit of U.S. patent application Ser. No. 17/099,772 titled “Vein Thromboembolism (VTE) Risk Assessment System” and filed Nov. 16, 2020, the contents of which are hereby incorporated by reference in their entirety.

### BACKGROUND

### Technical Field

The application is generally directed towards systems and methods for assessing pulmonary health for use with a handheld electronic device (HED) Example embodiments are related to, but not limited to, pulmonary health assessment systems for analyzing and detecting an unhealthy lung due to lung cancer, pulmonary embolism, and/or pulmonary fibrosis. Additional embodiments are related to but are not limited to, a system and method for analyzing and detecting vein thromboembolism (VTE) risk.

### Description of the Related Art

Artificial intelligence (AI) and machine learning (ML) enabled medicine and healthcare products are used to effectively support patients with long-term conditions at home. In particular, AI can be useful in the absence of conclusive evidence of decision-making AI can help to analyze continuous data received from patients infected and not infected with pulmonary health conditions in real-time to understand and predict if the pulmonary health condition is present or evolving in the patient's body. In a pandemic situation, the available number of healthcare professionals per patient is reduced to treat those patients affected by pulmonary health conditions. AI can help to identify those patients who need attention and channel them to healthcare professionals so they can focus on delivering healthcare.

Currently, pulmonary (relating to the lungs) diseases or disorders are common all over the world. The pulmonary diseases are classified into various categories such as breathing rhythm disorders, obstructive diseases, pulmonary congestion resulting from heart failure, restrictive diseases, infectious diseases, pulmonary vasculature disorders, pleural cavity disorders, and the like. Among the most widespread pathologies of the respiratory system can be identified as chronic obstructive pulmonary disease (COPD), tuberculosis, respiratory infections of the lower respiratory tract, oncology, as well as many others. Pulmonary diseases can be associated with a decrease in the total volume of exhaled airflow caused by a narrowing or blockage of the airways. Examples of COPD include asthma, emphysema, and bronchitis.

Further, pulmonary diseases may be caused by infectious agents such as viral and/or bacterial agents. Examples of infectious pulmonary diseases include pneumonia, tuberculosis, and bronchiectasis. Non-infectious pulmonary diseases include lung cancer and adult respiratory distress syndrome (ARDS), for example. In cases where pulmonary diseases can be detected and diagnosed at an early stage, the likelihood of successful treatment can be improved.

Many pulmonary conditions are analyzed by radiologists to determine and diagnose the likely causes of physical symptoms. Chest X-rays and/or Computed Tomography (CT) scans may be used by clinicians to gain a better understanding of what conditions may be present and to what extent. These methods can have problematic issues. Some disadvantages of Chest X-rays and CT scans are that patients may be exposed to radiation and in patients with significant kidney problems, the use of contrast material (dye) can be harmful. They can also be expensive to use. These methods can also be unsuitable for continuous and regular monitoring of patients' health conditions as well as for broad-scale screening purposes across a population for use in identifying and treating conditions early.

### SUMMARY

There is a need for pulmonary health assessment systems and methods that can provide a better patient experience by enabling the patients to self-monitor their lung conditions. There is also a need for an affordable and accurate screening tool that can help rule in patients who may benefit from early identification and treatments of certain conditions. For example, the survival rates of lung cancer patients have been shown to improve with early identification of the presence of this disease. Thus, there is a need for systems, devices, and methods that can accurately measure one or more different pulmonary conditions simultaneously while also providing data and processing for health assessments of such conditions. There is also a need to integrate such pulmonary health assessment systems and devices with computing devices that continue to increase in computing power and capability.

Additionally, AI and ML can help to diagnose and treat venous thromboembolism (VTE) which is caused by a thrombus (blood clot) formed within a vein and often leads to pulmonary embolism and pulmonary health issues. When a patient first experiences symptoms of VTE and begins to seek clinical treatment, the situation is very often quite far progressed and serious. It is therefore desirable to provide a system that can detect and monitor the patient continuously and detect in real-time the potential progression of VTE in at-risk patients such as patients in acute care and/or post-acute settings. Further, there is a need for a safe system that can utilize AI and ML to facilitate the patients to detect VTE in the absence of medical practitioners. There is furthermore a need for portable vein thromboembolism (VTE) risk assessment system that can aid in analyzing and detecting vein thromboembolism (VTE).

Systems and methods for assessing pulmonary health are provided, as shown in and/or described in connection with at least one of the figures.

One aspect of the present disclosure relates to a system for assessing pulmonary health. The system includes a handheld electronic device (HED); a casing; and at least one circuit board. The HED includes a display screen, a processor, and a software application. The casing has a shape adapted to secure a plurality of electronic components and the HED. The casing includes at least one diaphragm and a plurality of ECG electrodes that are placed on the outer surface of the casing. The ECG electrodes capture the electrophysiological data of the user. The circuit board is configured within the casing and electrically connected with the ECG electrodes and a microcontroller. The circuit board is further connected to at least one sound transducer and at least one Inertial Measurement Unit (IMU) sensor. The sound transducer captures pulmonary signals indicative of pulmonary health. The IMU sensor captures seismic and gyroscope signals indicative of the pulmonary health of the user and the orientation of the casing. The diaphragm enhances the pulmonary audio signals captured by the sound transducer. The microcontroller transmits the electrophysiological data received from the plurality of ECG electrodes, the sound transducer, and the IMU sensor to at least one of the HED and a computing device. The HED and computing device are configured to: receive, in one or more temporal windows, a representation of data from one or more of the following when positioned against the thoracic cavity of the user: the IMU sensor signals, the plurality of ECG electrodes signals, and the sound transducer signals; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the pulmonary health of the user and/or estimate a progression of a pulmonary health condition.

In an embodiment, the system includes a second electronic device worn by the user, wirelessly connected with the HED. The second electronic device includes one or more sensors and a wireless transceiver. The sensors collect health data including but not limited to photoplethysmography and/or accelerometer data from an upper limb region of the user. A wireless transceiver establishes a communication between the second electronic device and the computing device to transmit pulmonary health data therebetween. The computing device is configured to: detect, based on the classification model, the presence of indicators of pulmonary health condition, and estimate, using the indicators of the pulmonary health condition, based on the regression model, the severity of the pulmonary health condition. In an embodiment, the circuit board is further connected to at least one sound transducer that emits a noise that is reflected and sensed by one or more of the sound transducers. The reflected noise is utilized to provide an assessment of the user's pulmonary health. In an embodiment, the second electronic device may be held against the thoracic cage of the user, the back of the user, and/or the sternum of the user to emit sound into the body of the user.

In another embodiment, the system includes an additional sound transducer configured to emit soundwaves into the body of the user.

Additionally or alternatively, the circuit board is further connected to at least one transducer that emits vibrations that are reflected and sensed by one or more of the IMU transducers. The reflected vibrations are utilized to provide an assessment of the user's pulmonary health. Additionally or alternatively, the second electronic device may be held against the thoracic cage of the user, the back of the user, and/or the sternum of the user to emit vibrations into the body of the user.

Additionally or alternatively, the system includes an additional transducer configured to emit vibrations into the body of the user.

In an embodiment, the processor is configured to present one or more commands to determine the positioning of the casing on the user's body.

In an embodiment, the classification model is trained to detect an unhealthy lung caused by severe acute respiratory syndrome (SARS).

In an embodiment, the classification model is trained to classify an unhealthy lung caused by pulmonary congestion.

In an embodiment, the classification model is trained based on data received from one or more of the following: computerized tomography (CT) scans of the lung, X-ray of the chest, spirometer data, magnetic resonance imaging (MRI) data, and sound transducer data.

In an embodiment, the casing further includes a temperature sensor for detecting chest skin surface temperature resulting from variations in lung fluid levels. Changes in tissue from reduced oxygen levels which often correlate with lung congestion may furthermore cause changes in tissue temperature.

In some embodiments, the classification model is trained to classify abnormal pulmonary health activity arising from the plurality of parameters by deploying a pulmonary disease classification model. Abnormal pulmonary health may include but is not limited to chronic obstructive pulmonary disease (COPD) or any type of obstructive lung disease characterized by long-term breathing problems and poor airflow, pulmonary fibrosis, respiratory syndromes, increases in lung fluid levels and/or pulmonary embolism). Lung fluid levels often correlate with intracardiac pressures through the pulmonary arteries. Increased lung fluid levels may be detectable through an increase in density in the thoracic region which may cause noticeable differences in acoustic and/or seismic waveforms. Deteriorations in pulmonary health often correlate with deteriorations in cardiac health including but not limited to acute decompensation.

In an embodiment, the temperature sensor is a heat-sensing camera that detects variations in the skin area temperature resulting from variations in blood volume changes in the skin area in response to venous hemodynamic changes in the lower limb.

In an embodiment, the diaphragm includes an enhancer unit for enhancing the pulmonary audio signals including the ability to amplify low-frequency pulmonary soundwave signals.

In an embodiment, the HED further includes a battery configured to supply electrical power to the circuit board.

In an embodiment, the casing further includes a lens configured to envelop a camera of the HED

In an embodiment, the casing further includes the lens configured to block external light when the HED shines a light onto the skin of the user; wherein the light is used to help with the recording of one or more images of the skin of the user, and wherein the one or more images are analyzed based on machine learning to provide insights into the pulmonary health of the user.

In an embodiment, the data indicating a high severity of a pulmonary health condition triggers a message transmission to a healthcare professional.

In an embodiment, the classification model is trained to classify an unhealthy lung caused by lung cancer.

In an embodiment, the processor is configured to provide instructions to the user regarding the management of the user's disease.

In an embodiment, the casing further includes one or more of the following: a magnet, radiofrequency coils, and a gradient coil.

In an embodiment, the casing includes two or more IMU sensors, two or more sound transducers, and two or more PPG sensors.

In an embodiment, the processor prompts the user to log in with a username.

In an embodiment, the casing is placed in a lower limb region of the user to collect vein thromboembolism (VTE) risk data therefrom.

In an embodiment, the processor is configured to display a result of the calculation on a display screen of a computing device.

In an embodiment, the pulmonary health condition includes lung fluid levels.

In an embodiment, the circuit board is further connected to at least one Photoplethysmography (PPG) sensor for measuring blood flow patterns indicative of the pulmonary health of the user.

An aspect of the present disclosure relates to a handheld electronic device (HED) for assessing pulmonary health. The handheld electronic device (HED) includes at least two ECG electrodes; at least one sound transducer; at least one Inertial Measurement Unit (IMU) sensor; and a microcontroller. The ECG electrodes are placed on the outer surface of the HED The ECG electrodes are configured to capture the electrophysiological data of the user. The sound transducer captures soundwave signals indicative of the pulmonary health of the user. The Inertial Measurement Unit (IMU) sensor captures seismic and gyroscope signals indicative of the pulmonary health of the user and the orientation of the HED The microcontroller transmits electrophysiological data received from the ECG electrodes, the sound transducer, and the IMU sensor to at least one of the HED and a computing device. At least one of the HED and the computing device is configured to: receive, in one or more temporal windows, a representation of data from one or more of the following when positioned against the thoracic cavity of the user: the IMU sensor signals, the plurality of ECG electrodes signals, and the sound transducer signals; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the pulmonary health of the user and/or estimate a progression of a pulmonary health condition.

In an embodiment, the HED includes two or more IMU sensors, two or more sound transducers, and two or more PPG sensors.

In an embodiment, the pulmonary health condition lung fluid levels. In an embodiment, the handheld electronic device (HED) includes at least one Photoplethysmography (PPG) sensor to measure blood flow patterns indicative of the pulmonary health of the user.

An aspect of the present disclosure relates to a method for assessing pulmonary health. The method includes a step of capturing electrophysiological data of the user through a plurality of ECG electrodes placed on an outer surface of a casing. The casing has a shape adapted to secure a plurality of electronic components and a handheld electronic device (HED). The casing includes at least one circuit board and at least one diaphragm. The circuit board is connected to one or more of the ECG electrodes; at least one microcontroller; at least one sound transducer; and at least one Inertial Measurement Unit (IMU) sensor. The method includes a step of capturing pulmonary signals indicative of pulmonary health through the sound transducer. The method includes a step of capturing seismic and gyroscope signals indicative of the pulmonary health of the user and orientation of the casing through the Inertial Measurement Unit (IMU) sensor. The method includes a step of enhancing the pulmonary audio signals captured by the sound transducer through the diaphragm. The microcontroller is configured to transmit electrophysiological data received from the plurality of ECG electrodes, the sound transducer, and the IMU sensor to at least one of the HED and a computing device. At least one of the HED and a computing device is configured to: receive, in one or more temporal windows, a representation of data from one or more of the following when positioned against the thoracic cavity of the user: the IMU sensor signals, the plurality of ECG electrodes signals, and the sound transducer signals; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the pulmonary health of the user and/or estimate a progression of a pulmonary health condition.

In an embodiment, the circuit board is further connected to at least one sound transducer that emits a noise that is reflected and sensed by one or more of the sound transducers, wherein the reflected noise is utilized to provide an assessment of the user's pulmonary health.

In an embodiment, the circuit board is further connected to at least one transducer that emits a vibration that is reflected and sensed by one or more of the IMU sensors, wherein the reflected vibrations are utilized to provide an assessment of the user's pulmonary health. Additionally or alternatively, transmitted vibrations may be used with a transducer on the distal or adjacent sides of the body from the proximally sensed side.

In an embodiment, the processor is configured to present one or more commands to determine the positioning of the casing on the user's body.

In an embodiment, the classification model is trained to classify an unhealthy lung caused by severe acute respiratory syndrome (SARS).

In an embodiment, the classification model is trained to classify an unhealthy lung caused by pulmonary congestion.

In an embodiment, the classification model is trained based on data received from one or more of the following: computerized tomography (CT) scans of the lung, X-ray of the chest, spirometer data, magnetic resonance imaging (MRI) data, and sound transducer data.

In an embodiment, the casing further includes a temperature sensor for detecting chest skin surface temperature.

In an embodiment, the temperature sensor is a heat-sensing camera that detects variations in the skin area temperature resulting from variations in blood volume changes in the skin area in response to venous hemodynamic changes in the lower limb.

In an embodiment, the diaphragm includes an enhancer unit for enhancing the pulmonary audio signals including the ability to amplify low-frequency pulmonary soundwave signals.

In an embodiment, the HED further includes a battery configured to supply electrical power to the circuit board.

In an embodiment, the casing further includes a lens configured to envelop a camera of the HED.

In an embodiment, the casing further includes the lens configured to block external light when the HED shines a light onto the skin of the user. The light is used to help with the recording of one or more images of the skin of the user, and wherein the one or more images are analyzed based on machine learning to provide insights into the pulmonary health of the user.

In an embodiment, the data indicating a high severity of a pulmonary health condition triggers a message transmission to a healthcare professional.

In an embodiment, the classification model is trained to classify an unhealthy lung caused by lung cancer.

In an embodiment, the processor is configured to provide instructions to the user regarding the management of the user's disease.

In an embodiment, the casing further includes one or more of the following: a magnet, radiofrequency coils, and a gradient coil.

In an embodiment, the casing includes two or more IMU sensors, two or more sound transducers, and two or more PPG sensors.

In an embodiment, the processor prompts the user to log in with a username.

In an embodiment, the casing is placed in a lower limb region of the user to collect vein thromboembolism (VTE) risk data therefrom.

In an embodiment, the processor is configured to display a result of the calculation on a display screen of a computing device.

In an embodiment, the pulmonary health condition includes lung fluid levels.

In an embodiment, the circuit board is further connected to at least one Photoplethysmography (PPG) sensor for measuring blood flow patterns indicative of the pulmonary health of the user.

Accordingly, example embodiments enable non-invasive and affordable methods of health assessment to identify conditions of the lungs aiding in the treatment and preventive care for patients.

Other features of the example embodiments will be apparent from the drawings and from the detailed description that follows.

### BRIEF DESCRIPTION OF THE DRAWINGS

The accompanying drawings illustrate the embodiments of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale.

Various embodiments will hereinafter be described in accordance with the appended drawings, which are provided to illustrate, not limit, the scope, wherein similar designations denote similar elements, and in which:

FIG. 1 illustrates a perspective view of the various components of an example system for assessing pulmonary health;

FIG. 2 illustrates a front view of the various components of the circuit board of the casing;

FIG. 3 illustrates an example network implementation of the above embodiment;

FIG. 4 illustrates a perspective view of communication between the casing and a second electronic device;

FIG. 5 illustrates a perspective view of the casing embodiment connected with the HED;

FIG. 6a illustrates example placements of the casing on the patient's chest and rib cage;

FIG. 6b illustrates an example placement of the casing on the back portion of the patient's body;

FIG. 7 illustrates a perspective view of the unhealthy lung due to severe acute respiratory syndrome (SARS) in conjunction with an example graphical representation of the acoustic signal level;

FIGS. 8a-8c illustrate a plurality of example user interfaces and instructions presented on the HED related to the usage of the casing;

FIGS. 9a-9c illustrate a plurality of example user interfaces used with operations performed by mobile applications on the HED;

FIG. 10 illustrates an example user interface depicting the user's personal information and pulmonary diagnostic information;

FIG. 11 illustrates a flow diagram of using data for model training;

FIG. 12 illustrates example positions of the casing over a rear view of leg to detect or classify deep vein thrombosis (part of vein thromboembolism (VTE));

FIG. 13 illustrates a cross sectional view of a vein with deep vein thrombosis (part of vein thromboembolism (VTE));

FIG. 14 illustrates an example graphical representation of an example IMU and/or microphone signal level;

FIG. 15 illustrates an example user interface detailing a user's personal information and VTE diagnostic information; and

FIG. 16 illustrates a different example embodiment of the system with a sound transducer configured to emit soundwaves in the thoracic region of the user.

### DETAILED DESCRIPTION

The present disclosure is best understood with reference to the detailed figures and description set forth herein. Various embodiments of the present systems, devices, and methods have been discussed with reference to the figures. However, those skilled in the art will readily appreciate that the detailed description provided herein including the figures are presented for explanatory purposes and the embodiments extend beyond the currently described embodiments. For instance, the teachings and results presented in any particular described application may yield multiple alternative approaches and may be implemented in any suitable manner.

The described embodiments may be implemented manually, automatically, and/or a combination of thereof. The term “method” refers to manners, means, techniques, and procedures for accomplishing any task including, but not limited to, those manners, means, techniques, and procedures either known to the person skilled in the art or readily developed from existing manners, means, techniques and procedures by practitioners of the art to which the embodiments pertain. Persons skilled in the art will envision many other variations that are within the scope of the claimed subject matter.

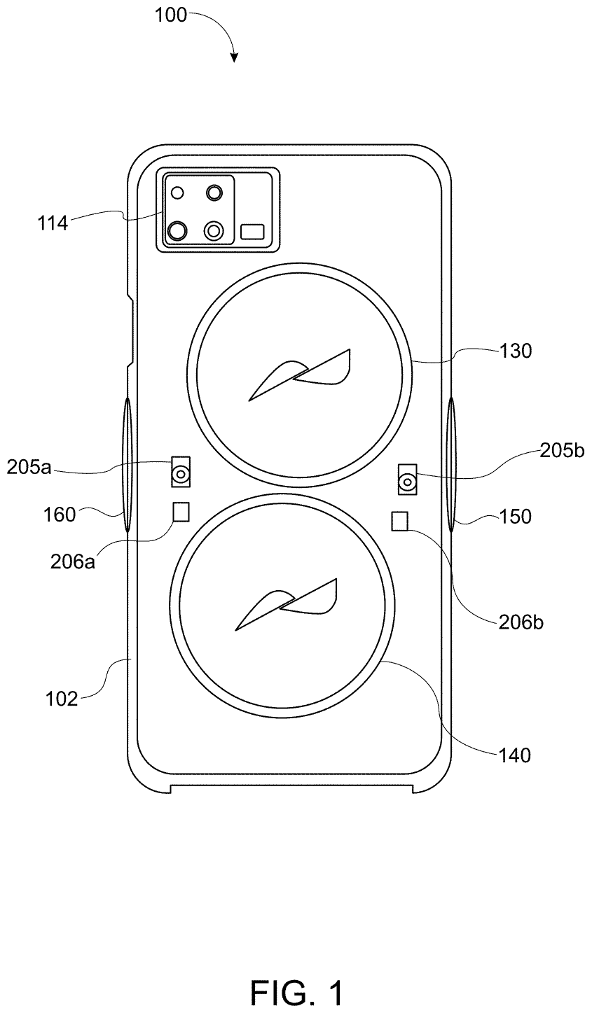

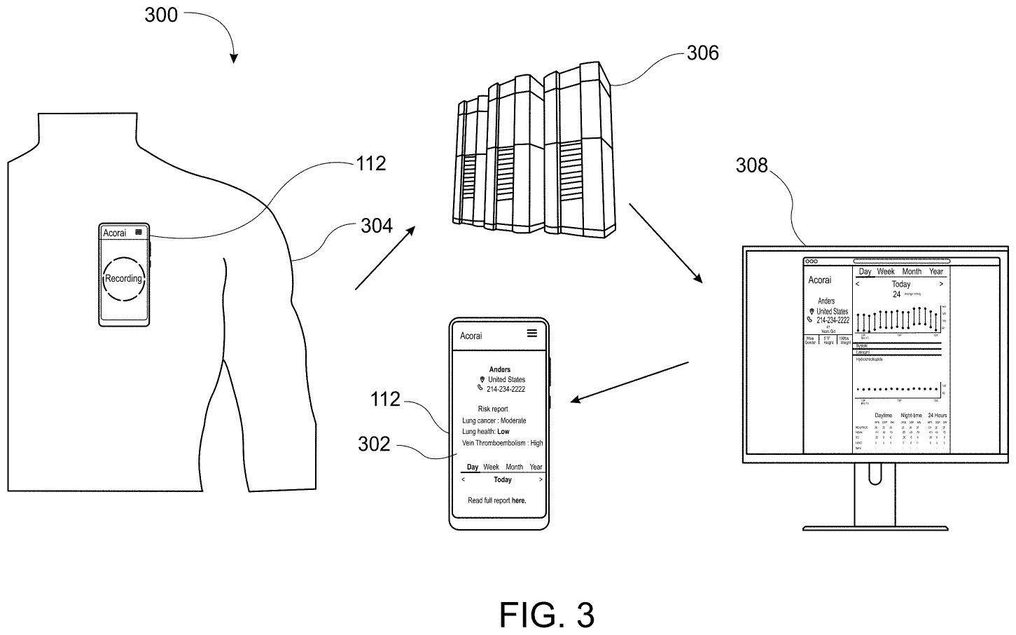

FIG. 1 illustrates a perspective view of an embodiment 100 which includes various components of the present system for assessing pulmonary health in accordance with at least one embodiment of the claimed subject matter. Embodiment 100 includes a handheld electronic device (HED) 112 (shown in FIG. 3), a casing 102, and at least one circuit board 110. The casing 102 having a shape adapted to secure a plurality of electronic components and the handheld electronic device (HED) 112 can be positioned with at least a portion of the casing 102. In many embodiments, the positioning of the HED 112 with a casing 102 may encompass or include the positioning of the HED 112 within all or a portion of the casing 102. In many embodiments, the shape of the casing 102 can be adapted for any suitable HED 112, for example, a mobile phone, smartphone, and/or any electronic device with a digital display screen. In these embodiments, the HED 112 can be accommodated within and positioned securely within all or a portion of the casing 102. The HED 112 includes a display screen, a processor, and a software application.

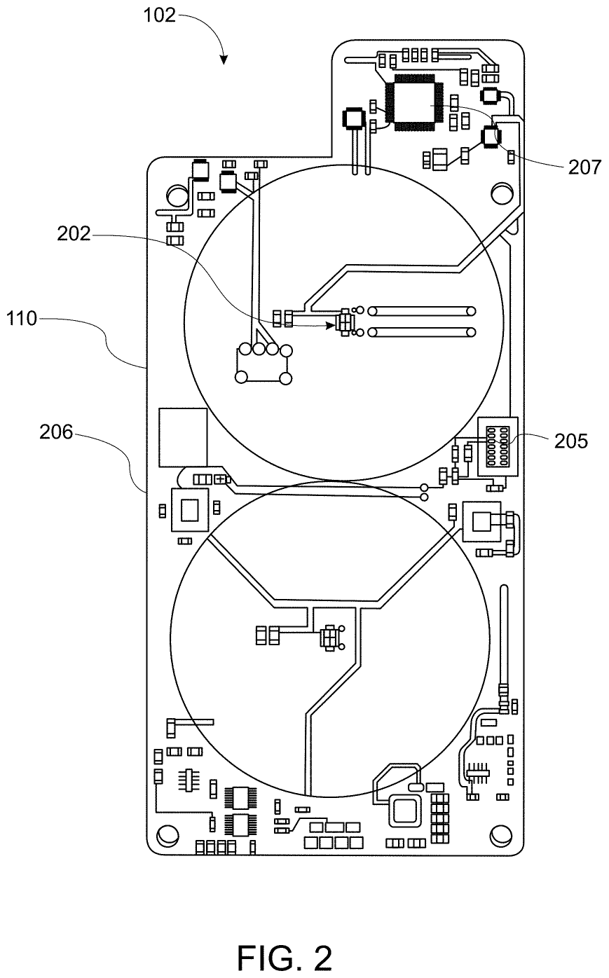

In these embodiments, casing 102 includes a plurality of electrodes 150, and 160. As shown in FIG. 2, the electrodes include a first ECG electrode 150 and a second ECG electrode 160. The first ECG electrode 150 and the second ECG electrode 160 are placed on the outer surface of casing 102. The first ECG electrode 150 and the second ECG electrode 160 may be placed on each side of the casing 102 to facilitate a thumb and fingers of a user to be placed on the casing 102 with the casing 102 having a shape that is adapted to secure the HED 112 or be placed on the side and level as the remaining sensors of the casing. In these embodiments, the ECG electrodes 150 and 160 are configured to capture the electrophysiological data of the user. In some embodiments, some or all ECG electrodes may be placed on the same side of the casing to ensure that at least two of the electrodes are in physical contact with the thoracic cavity of the user.

FIG. 2 illustrates an exploded view of the various components of the casing 102 in accordance with embodiments of the claimed subject matter and can be viewed in conjunction with FIG. 1. As shown in FIG. 2, the circuit board 110, configured within casing 102, is electrically connected with the plurality of electrodes 150 and 160. In these embodiments, the circuit board 110 includes a sound sensor 202, one or more Photoplethysmography (PPG) sensors 205a and 205b, one or more Inertial Measurement Unit (IMU) sensors 206a and 206b, and a microcontroller (not shown.) The sound sensor 202 captures pulmonary audio signals indicative of the pulmonary health of the user. According to an embodiment herein, the sound transducer 202 is operable to transmit and receive soundwaves. The diaphragm(s) 130 140 enhances the pulmonary audio signals captured by the sound transducer sensor. In many embodiments, the diaphragm includes an enhancer unit such as a bell-like object to amplify low-frequency auscultation signals pertaining to the pulmonary audio signals. The bell-like object may be any suitable enhancing element known to those skilled in the art. In some embodiments, the diaphragm may be configured as a tube structure to enhance low-frequency sounds although the tube structure may be configured in any other suitable form, such as in a stethoscope type configuration. According to an embodiment herein, the diaphragm 130 140 can be viewed as integrated with the sound transducer and operates as a plurality of piezoelectric plates.

In these embodiments, the PPG sensor 205 measures pulmonary capillary blood flow. In some embodiments, the PPG sensor 205 generates infrared (IR) light to measure the pulmonary capillary blood flow. In these embodiments, the PPG sensor 205 is a non-invasive, inexpensive, and convenient diagnostic tool to measure oxygen saturation, blood pressure, and cardiac output. In many embodiments, the PPG sensor 205 is placed at the top right of the casing 102 and may be connected to one or more additional microcontrollers.

Embodiments also include Inertial Measurement Unit (IMU) sensors (206a and 206b) for capturing seismic and auscultation signals that are indicative of the pulmonary health of the user. The IMU sensors 206 and 206b include at least one IMU sensor signal enhancing material 116 that amplifies seismic and auscultation signals. Examples of the IMU sensor signal enhancing material include, but are not limited to, sound absorbers made from porous materials, micro-perforated plates, and micro-perforated panel absorbers backed with mechanical impedance plates where the backed cavity is limited as well as combinations thereof. The microcontroller transmits pulmonary health data received from the plurality of electrodes 150 and 160, the sound sensor 202, the PPG sensors 205a and 205b, and the IMU sensors 206a and 206b to the HED and a computing device such as the server 306 illustrated in FIG. 3.

In many embodiments, casing 102 includes a lens 114 configured to envelop the camera of the HED 112. The lens 114 may be configured to cover all or a portion of the camera of the HED 112. In some of these embodiments, the lens is configured to block all or a portion of the external light when the HED 112 shines light into the skin of the patient and simultaneously captures images or records video of the skin. In some embodiments, video in addition to or instead of one or more images may be recorded by the camera.

The camera is used to record one or more images of the user's skin and the one or more images are analyzed by the system using machine learning to aid in providing insights into the pulmonary health of the user based on differences in the user's tissue color detected and applying machine learning such as applying one or more image recognition machine learning models with the one or more images to aid in providing insights into lung conditions.

In an embodiment, casing 102 further includes a temperature sensor for detecting chest skin surface temperature. According to an embodiment herein, the temperature sensor 207 is a heat-sensing camera that detects variations in the skin area temperature resulting from variations in blood volume changes in the skin area in response to venous hemodynamic changes in the lower limb.

In several embodiments, casing 102 includes a battery configured to supply electrical power to the circuit board 110. In some instances, the circuit board may receive power from an external source. The casing 102 may connect with the HED 112 through a power cable or any other suitable connection. In several embodiments, casing 102 includes one or more additional sensors such as seismic and sound transducer sensors which can be used to facilitate the identification of common ambient environmental noise unrelated to the patient's pulmonary health. In some embodiments, the presence of data indicating a high severity of a pulmonary health condition triggers the transmission of one or more messages to a healthcare professional or other health monitor.

In many of the embodiments, the user is guided by the HED 112 through instruction, for example, visual or audio instruction, as to where to place the device on the user's body. In some embodiments, the instructions may indicate corrective actions the user can take to optimally place the HED 112 on a user. Additionally, in many embodiments, previously collected sensor data is used to identify a user's unique physiological markers.

FIG. 3 illustrates a network implementation of the present pulmonary health assessment system 300, in accordance with embodiments of the claimed subject matter. In several embodiments, the HED 112 includes a display screen 302 for displaying pulmonary diagnostic information using pulmonary health data received from the microcontroller. In many embodiments, casing 102 is configured with the HED 112 positioned against the chest of user 304 so that it can capture the user's pulmonary health data. In other embodiments, casing 102 is configured to capture the user's pulmonary health data when the HED 112 is positioned against the back of the user and/or the thoracic cage of the user.

In some embodiments, the HED 112 is positioned and secured within casing 102. The HED 112 includes a processor to execute a plurality of instructions according to the requirements of one or more pulmonary monitoring applications. In many embodiments, the processor is configured to display one or more commands, for example, audio or visual commands, so that the user or a third party can position the casing 102 against the chest of the user. Similarly, the processor can also instruct a user or a third party to position the HED 112 to optimize the sensor data acquisition. In many embodiments, the processor commands include an instruction to the user or a third party operating the HED 112 to position and hold the casing 102 against the user in a particular manner such as holding it against the user with one hand.

In many embodiments, the classification model is trained to classify an unhealthy lung, for example, a user's lung affected by severe acute respiratory syndrome (SARS). The classification model is trained based on data received from one or more computerized tomography (CT) scans of the lung, X-ray of the chest, and spirometer data, magnetic resonance imaging (MM) data, and ultrasound data. In some embodiments, the spirometer data is collected by a spirometer. The spirometer measures the volume of air inspired and expired by the lungs as well as ventilation, the movement of air into and out of the lungs. According to several embodiments, at least one or more of the HED 112 and the computing device 306 is configured to receive, in one or more temporal windows, a representation of one or more of the following: the IMU sensor, the plurality of electrodes, the PPG sensor, and the sound transducer sensor signal recorded by the casing 112.

In these embodiments, at least one or more of the HED 112 and the computing device 306 is configured to detect features of the IMU sensor, the PPG sensor, and the sound transducer sensor from at least one or more portions of the received representations falling within each of the one or more temporal windows.

The HED 112 and computing device 306 are additionally configured to identify patterns of the features of respective sensors from within one or more portions based on at least one of the following: a classification model and a regression model.

The computing device 306 is configured to calculate, based on the identified patterns, a probability of whether one or more portions corresponding to a problem with the pulmonary health of the user. In many embodiments, the classification model is trained to classify an unhealthy lung due to lung cancer, pulmonary fibrosis, pulmonary congestion, and/or severe acute respiratory diseases. In many embodiments, pulmonary congestion may arise from heart failure and increased intracardiac pressures of the user. The pulmonary congestion assessment may be derived from a probability of having pulmonary congestion and/or as a percentage level of fluid present within the lung. In an embodiment, a pulmonary congestion index may be created to classify the level of congestion according to different groups of congestion, where some groups represent a range of higher congestion levels that may indicate a higher risk of hospitalization, mortality and/or adverse event risk. In an embodiment, said pulmonary congestion index may provide information on whether a pulmonary congestion is being caused by problems associated with the right side of the heart and/or the left side of the heart and/or the pulmonary artery and/or other cardiac diseases. In an embodiment, the regression model and the classification model that estimates pulmonary congestion may be trained using lung fluid levels from image data including but not limited to a chest X-ray, CT-scan, MM and/or echocardiography. In an embodiment, the pulmonary congestion regression and classification models may be trained using patient outcome-based data, including but not limited to heart failure hospitalizations and/or mortality events of one or more users.

In many embodiments, the processor is configured to transmit the data indicative of pulmonary health from the handheld electronic device 112 to server 306 over a network; and store the data in server 306 for subsequent analysis by a clinician. Examples of the network could be one or more networks, or a combination of a local area network and a wide area network, such as the internet, using any suitable physical or wireless connections, for example, Wi-Fi, Ethernet, and Bluetooth connections. One or more wireless networks may be any network known to those skilled in the art, including by not limited to, a GSM, 3G, 4G, and a 5G network. In some embodiments, the processor is configured to transmit the data indicative of pulmonary function from the HED 112 to a clinician computing device 308 via the internet for use with remote diagnostic analysis and processes such as machine learning.

In many embodiments, the clinician computing device 308 performs risk analysis which can be presented in any desired visual and/or audio formats using the mobile application of the HED 112. Also, in many of the embodiments, the classification model is trained to classify abnormal lung activity. In several embodiments, the microcontroller utilizes a de-noising algorithm, for example using TensorFlow Lite which includes a machine learning library.

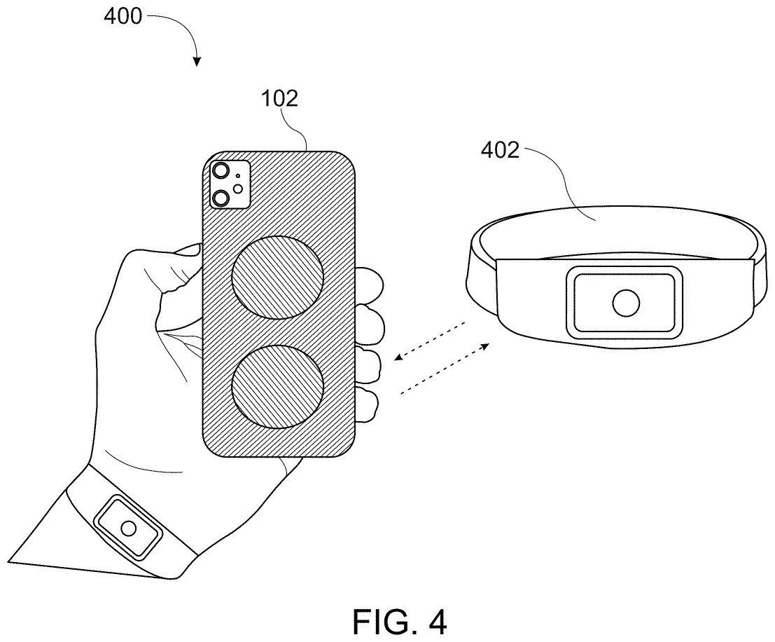

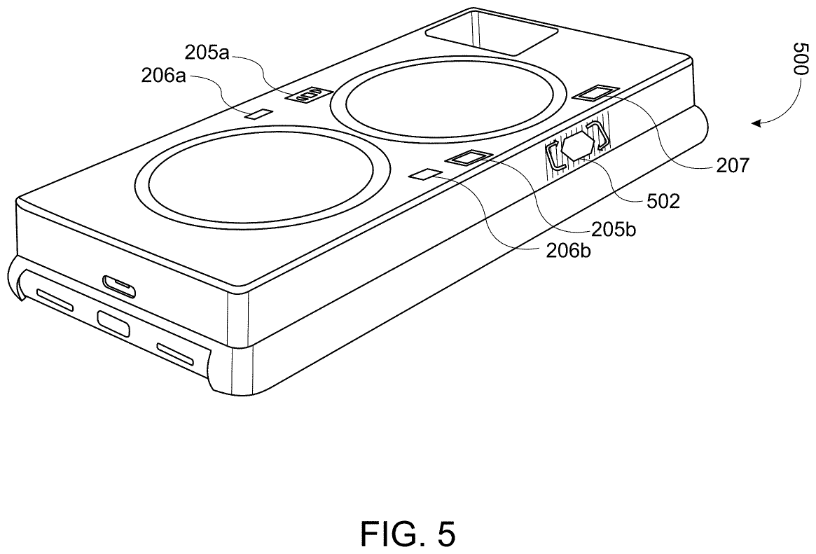

FIG. 4 illustrates a perspective view of a system 400 having communication between the casing 102 and a second electronic device 402, in accordance with several embodiments. In some embodiments, the system includes a second electronic device 402 that includes a wireless transceiver and an application. In an embodiment, the second electronic device 402 is wearable and wirelessly connected with the HED 112. The wireless transceiver receives pulmonary health data from casing 102 and establishes a communication with the server for transmission of pulmonary health data there-between. The HED-based application is programmable to transmit diagnostic information derived from the pulmonary health data received by the wireless transceiver. In some embodiments, the second electronic device 402 is a wearable device that can connect wirelessly with the handheld electronic device 112. Examples of the second electronic device 402 include “smartphones”, “smartwatches,” PCs, tablets, wearables, wearable patches, wearable remote monitoring solutions, or handheld computers that can download and examine data in real-time and/or in a different temporal segment. The second electronic device 402 may be a handheld electronic device. FIG. 5 illustrates a side view 500 of the casing 102 in accordance with embodiments of the claimed subject matter. The casing 102 includes a button 502 which can be used to initiate the operation (in/out) of a diaphragm 130.

Many embodiments include various sensors employing different technologies allowing for robustness across different recording environments and patients. For instance, in an environment with high soundwave noise, the classification model and/or regression model may be trained to emphasize visual, seismic, and/or electrophysiological sensors. In another example, wherein the embodiment is used with a darker-skinned patient wherein light is less able to penetrate adequately the skin, the classification model and/or regression model may be trained to emphasize sensors pertaining to seismic, audio, and/or electrophysiological sensors.

In other embodiments, the classification model is trained to classify the presence of a deficiency in a lung caused by a pulmonary embolism. Typically, pulmonary embolism is a blood clot in the lung that occurs when a clot in another part of the body (often the leg or arm) moves through the bloodstream and becomes lodged in the blood vessels of the lung. Pulmonary embolism and deep vein thrombosis (DVT) are interrelated diseases. Deep vein thrombosis can be very serious because blood clots in your veins can break loose, travel through your bloodstream, and get stuck in your lungs, blocking blood flow (pulmonary embolism). However, pulmonary embolism can occur without evidence of DVT. In some of the embodiments, the processor is configured to provide instructions pertaining to the management of the user's disease. Also, in many of the embodiments, casing 102 includes an ultrasound transducer, a magnet, radiofrequency coils, and a gradient coil. In many embodiments, casing 102 is configured to capture pulmonary health data of the user when positioned against the thoracic cavity and/or back of the user.

According to some embodiments, a physiological identification system includes a pulmonary health assessment device that utilizes a plurality of sensors and a computing device. In these embodiments, the computing device is configured to determine the identity of a user based on a plurality of unique physiological features of a user based on data from the pulmonary health assessment device's previous measurements. In many of these embodiments, the sensors include one or more of the following: a sound transducer sensor, at least one Photoplethysmography (PPG) sensor, at least one Inertial Measurement Unit (IMU) sensor, and an ultrasound transducer.

According to many embodiments of the health assessment device placement calibration system, a pulmonary health assessment device is equipped with a plurality of sensors and a display (screen) configured with one or more computing devices to display a plurality of instructions. Other embodiments may include a speaker in addition to a screen to broadcast audio instructions in addition to or instead of visual instructions. In some embodiments, the pulmonary assessment device is configured to perform the steps of determining the placement of said health assessment device based on unique physiological features of a user pertaining to one or more previous measurements from the pulmonary health assessment device and provide instructions of placement of the health assessment device through the display screen and/or the speaker. Other embodiments may include a touch instruction such as with the use of braille for visually impaired users. In many embodiments, the health assessment device placement calibration system includes one or more additional handheld electronic devices that transmit data to one or more computing devices to indicate the position of the health assessment device on the user's body. The one or more additional handheld electronic devices may employ a plurality of inertial measurement unit sensors to indicate the location, angle, and/or stationarity of the user.

Many of the embodiments allow the casing 102 to operate without a battery using direct power from an electronic device allowing for more space within the casing 102 so that larger and more powerful sensors may be used with the embodiments leading to enhanced data collection quality and accuracy. The absence of a battery may also help reduce the amount of electrical interference relating to the use of the casing's sensors. These embodiments allow for more powerful devices that alleviate the need to have multiple pulmonary health assessment devices to be able to identify a number of different lung conditions. Using a single device that can accurately analyze a number of lung conditions instead of multiple devices, the patient experience can be substantially improved helping ease the patient's anxiety and lead to more willingness to use the device for regular monitoring.

The utilization of the HED 112 with the casing 102 allows greater accuracy and efficiency of data transmission. Data relating to external noise, positioning, and movement of the HED 112 (and the attached casing 102) can be measured using the HED's internal accelerometer, microphonic and other sensors. Additional sensors may be added to increase the data input for additional external information. This measured data can be used to help remove noise from other collected health-related data so that analysis can be made on data that is most relevant to conditions pertaining to the pulmonary health of the user. In many embodiments, the HED 112 internal accelerometer and sound transducer sensor act as an acoustic sensor that provides an acoustic signal conveying information associated with internal respiratory sounds. In these embodiments, the acoustic sensor can sense tissue vibration.

Embodiments using smartphones allow a simplified means for a user to monitor their health. The user can carry the embodiments throughout the day and night and the casing 102 can also function as a protective barrier against breakage, surface scratching, and damaging environmental hazards such as water. Another benefit of the embodiments is the use of a single device instead of multiple devices for self-health monitoring which can also reduce the likelihood of misplacing the device. The embodiments also allow the use of battery power instead of or in addition to power from being wired to outlets allowing users to charge the embodiments at their conveniences such as at regular times during the day or night. Another benefit of the embodiments is the ability of a user to record at standardized time intervals.

In use, a patient may use the HED's one or more internal alarm clocks to remind the user as well as prompt the user to perform data recording functions using the embodiments. In many embodiments, the user can take readings at approximately the same time each day to allow the data collection to be performed during similar recording environments leading to a more standardized data collection to further aid in the reduction of noise in the collected data.

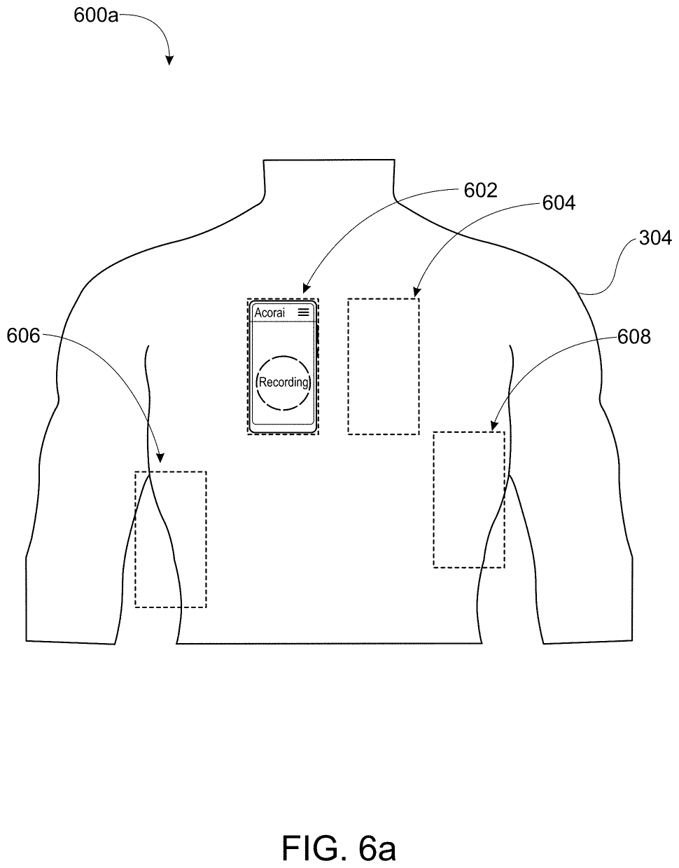

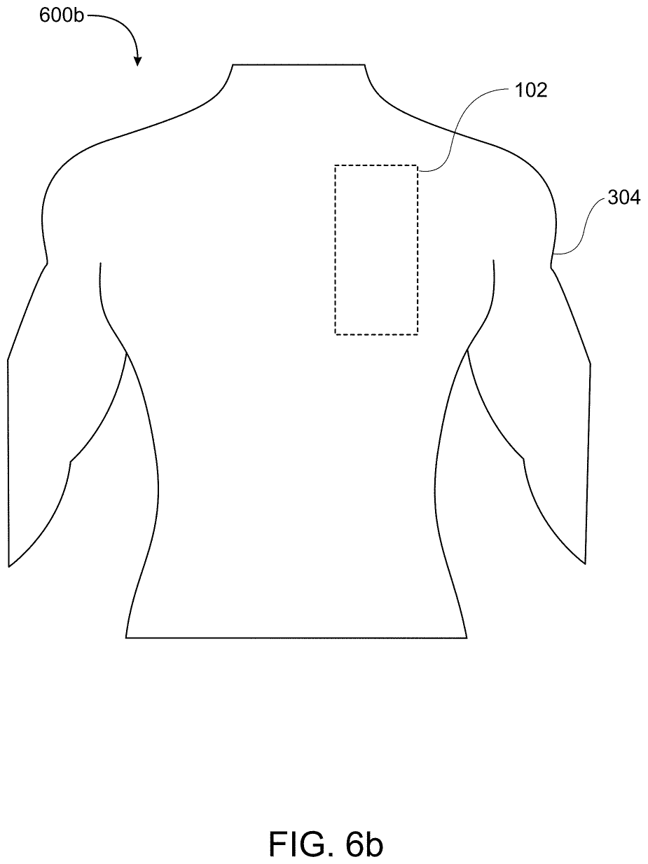

FIG. 6a illustrates a perspective view 600a of the placement of casing 102 on the patient's chest and rib cage in accordance with at least one embodiment. FIG. 6a is explained in conjunction with FIG. 1. As shown in FIG. 6a, casing 102 is placed centrally on the patient's chest. In many of the embodiments, the software application executed on the handheld electronic device (or a remote server) may be used to direct the user 304 of the casing 102 to position or correct the placement of the casing 102 on the chest of the user 304 or another person. Audio, visual, or tactile instructions or directions originating from the embodiment may include a first step 602 of placing the casing 102 in a position about a finger length's distance below the patient's right collarbone, alongside the sternum, and then in a second step 604, placing the casing 102 in a position about a finger length's distance below the patient's left collarbone alongside the sternum.

Next, in a third step 606, the user is instructed to place the casing 102 on the exterior of the right rib cage of the patient and, in a fourth step 608, the user is instructed to place the casing on the outer surface at the left rib cage of the patient so that steps 602 through 608 allow the embodiments to capture the user's pulmonary health data. FIG. 6b illustrates a perspective view 600b of the placement of the casing 102 on the back portion of the user's 304 body in accordance with at least one embodiment. In this position against the back of user 304, the embodiment can capture the pulmonary health data of user 304.

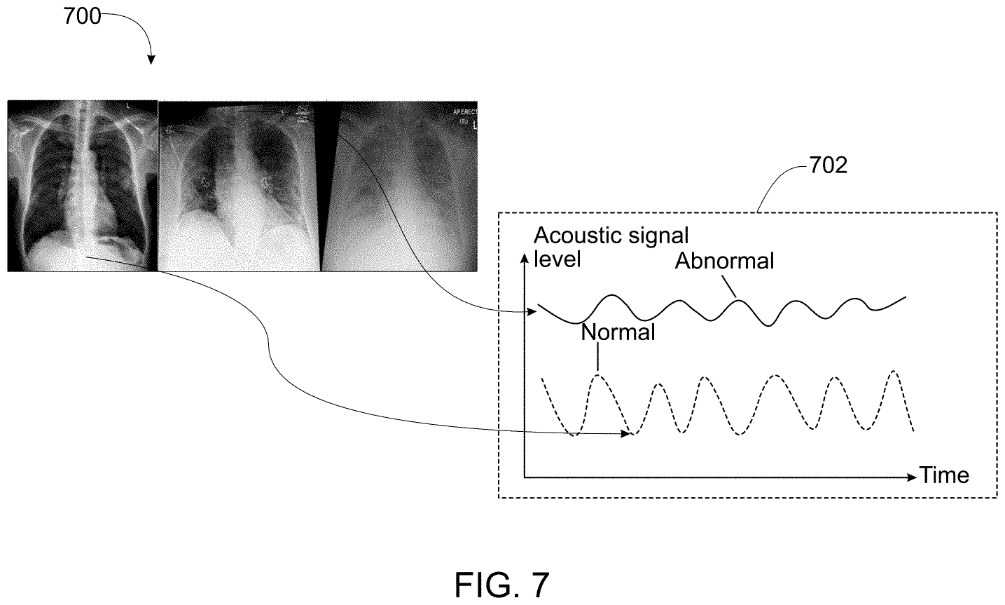

FIG. 7 illustrates a perspective view 700 of an exemplary image of lungs including an image of an unhealthy lung affected by severe acute respiratory syndrome (SARS) along with graphical representations 702 of acoustic signal levels in accordance with several embodiments. In these embodiments, the classification and/or regression models may be trained on data collected by the sensors using one or more of the following diagnostics: CT-scans of lungs (which is considered the “gold standard” by many practitioners), chest X-rays, Spirometers, MRIs, ultrasounds, as well as any other suitable diagnostic system or process providing information about the lung health of a patient.

In some embodiments, image-based indications of pulmonary health, including the above-described lung health information, may be used in any suitable manner to derive a clinical diagnosis probability. For example, information from an image may be used as a marker leading to a binary outcome of clinical diagnosis based on that image information. In another example, the image information may be used to calculate a percentage that could be used to represent the severity of the pulmonary health condition of the lung or lungs shown in the image. In many embodiments, the clinician's diagnoses may be used to train the models alone or in conjunction with other methods. In an embodiment, the pulmonary health condition includes lung fluid levels.

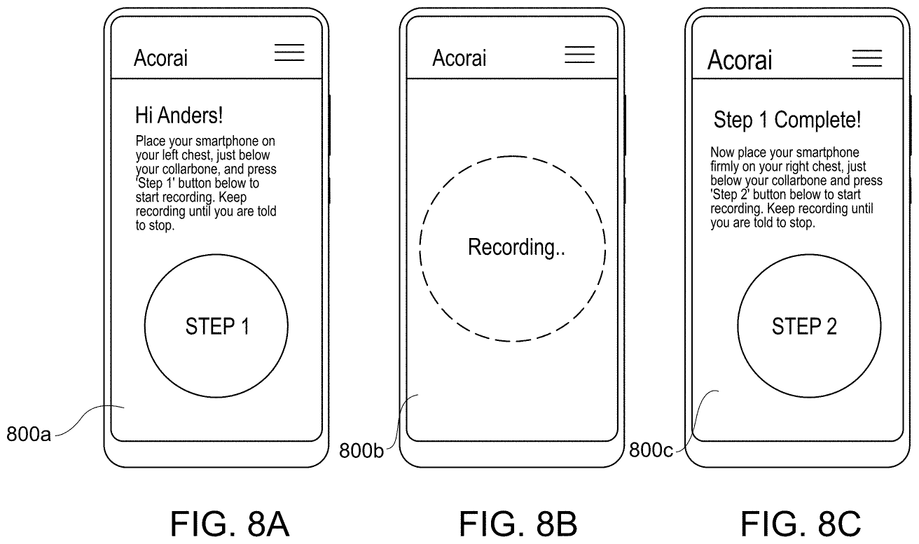

FIGS. 8a-8c illustrate a plurality of user interfaces 800a, 800b, and 800c depicting a plurality of visual directions pertaining to the usage of the casing 102 in accordance with many embodiments. The user interface 800a depicts an exemplary first step visual instructing the user to place the HED a finger below the patient's left collarbone and press the ‘Step 1’ button shown on the lower portion of the HED screen to start the recording which is shown in process in user interface 800b.

In these embodiments, the user maintains the position of the casing 102 allowing the embodiment to keep recording until the software application notifies/announces that the recording has stopped. After the recording has stopped, the user interface 800c depicts the visual text “Step 1 complete!” and further instructions for the user to first place the device firmly on his/her right chest positioned just below the collarbone and then press the ‘Step 2’ button below the text to start the recording. After the recording has stopped, the software application notifies the user that she or he can stop holding the device in the referenced recording position.

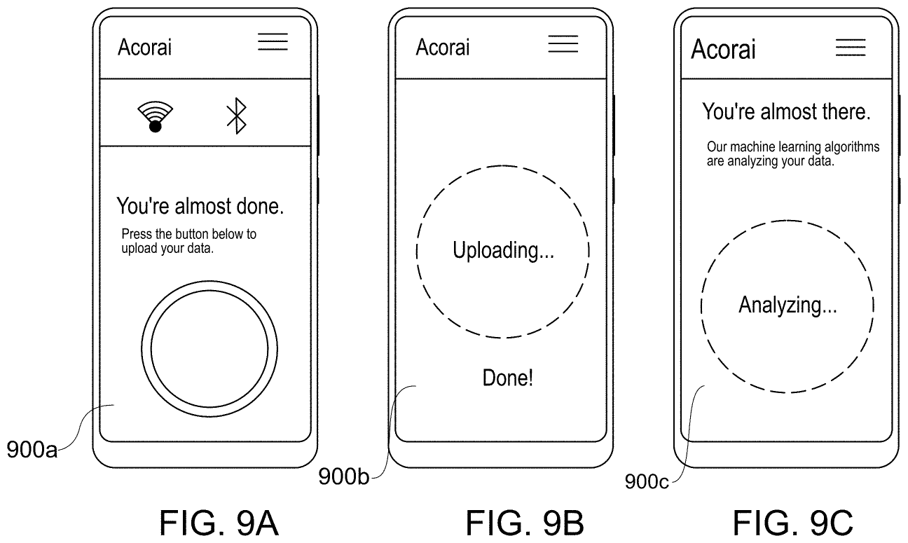

FIGS. 9a-9c illustrate user interfaces 900a, 900b, and 900c which depict operations performed by the mobile application in accordance with many embodiments. The user interface 900a shows the mobile application directing the user to begin an upload of the pulmonary health data received from the plurality of electrodes, the sound transducer sensor, and the IMU sensor. The user interface 900b depicts the pulmonary health data being uploaded and finishing the upload. The user interface 900c depicts the pending analysis of the pulmonary health data. The analysis performed using the pulmonary health data can aid in determining one or more preferred placements of the casing 102 on future sessions of the same user or positioning the casing 102 with other users. The analysis can also help with standardized data collection methods and techniques across a wide spectrum of issues and a number of users.

In many of the embodiments, the mobile application stores and can use previously stored data relating to certain physical features and/or characteristics associated with a patient's pulmonary signals (and potentially other sensor data associated with the user) which may be interpreted as the patient's unique “pulmonary ID”. This unique identifier can help ensure that the data collected can be verified as belonging to the patient and not someone else. The data may also be used to identify one of the multiple users that may be sharing the same device and it may also be used to determine when a patient has placed the casing 102 in a wrong position and prompt that patient to reposition the casing 102.

In some embodiments, the data collected through casing 102 may be combined with other data, for example, data sent from a wearable electronic device. By using additional data from other sources with the initial data, such as the data sent from a wearable sensor worn on the wrist and/or chest of a patient, enhanced accuracy may be achieved. For instance, the combination of data can help calculate pulse transit time and aid in the comparison of data between different parts of the body sensed at the same time. Some embodiments may use additional data from measurement readings of pulse oximetry devices as well as any other information related to pulmonary health. In many embodiments, the casing 102 may be used with a wireless charging station allowing one or more devices to be wirelessly charged. Also in many embodiments, a battery-less wearable may be connected to casing 102 and used for simultaneously recording data while drawing power directly or indirectly from casing 102.

After the data is recorded, it can be analyzed in the connected HED or another connected computing device, or the data may be uploaded to one or more servers where it can be analyzed. The data may also be analyzed by any combination of computing devices and servers. Embodiments may use any suitable methods for data analysis including, but not limited to, machine learning-based methods that are used to classify whether or not certain lung conditions are indicated in the data as being present. The machine learning methods used in the embodiments include, but are not limited to, decision tree-based machine learning methods, artificial neural networks, convolutional neural networks, logistic regression, naive Bayes, nearest neighbor, support vector machines, boosted tree learning methods, and deep learning methods.

In an additional embodiment, the user interface 900a shows the mobile application directing the user to begin an upload of the VTE risk data received from the plurality of electrodes, the sound transducer sensor, and the IMU sensor. According to an embodiment herein, the user interface 900b depicts the VTE risk data being uploaded and finishing the upload. Further, the user interface 900c depicts the pending analysis of the VTE risk data to figure out where it is most ideal to place the casing can furthermore help standardize data collection quality across different patient cohorts. The mobile application remembers certain physical features and/or characteristics associated with the patient's VTE risk signals which may be interpreted as the patient's “VTE risk ID.” This can ensure that the data collected can be verified as belonging to the patient and not someone else. It may furthermore indicate when the patient may have placed the casing in the wrong location and prompt the patient to reposition the casing.

In some embodiments, the data collected through the casing may be combined with data from a wearable electronic device. By combining data from other sources, such as a wearable sensor on the wrist of a patient, one may further enhance accuracy, for example by having the ability to calculate pulse transit time and compare data between different parts of the body at the same time. It may furthermore comprise measurements such as pulse oximetry and other VTE risk indications. In some embodiments, the casing 102 may be equipped with a wireless charging station to allow for wireless charging of other devices/wearables. A battery-less wearable may be connected to the casing and used to simultaneously record data, drawing power from the casing 102.

After the data is recorded, it can be analyzed in the connected HED or another connected computing device, or the data may be uploaded to one or more servers where it can be analyzed. The data may also be analyzed by any combination of computing devices and servers. Embodiments may use any suitable methods for data analysis including, but not limited to, machine learning-based methods that are used to classify whether or not certain lung conditions are indicated in the data as being present. The machine learning methods used in the embodiments include, but are not limited to, decision tree-based machine learning methods, artificial neural networks, convolutional neural networks, logistic regression, naive Bayes, nearest neighbor, support vector machines, boosted tree learning methods, and deep learning methods.

The classification and/or regression model may be trained on data collected by various sensors. Image-based indications of VTE risk, such as the aforementioned, may furthermore be a marker with a binary outcome of clinical diagnosis based on that picture, and/or given a percentage number indicating the severity of said VTE risk. The clinician's diagnoses may furthermore be used as the gold standard to train the models.

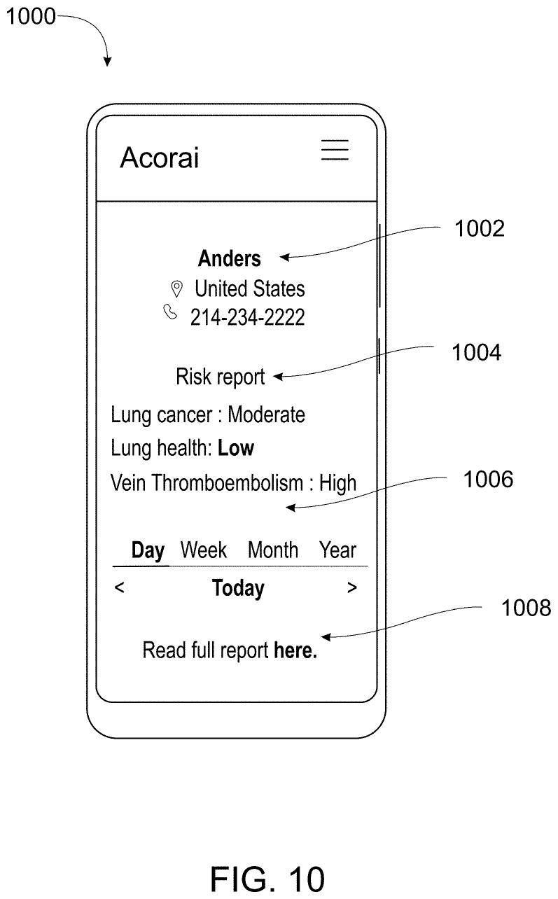

FIG. 10 illustrates a user interface 1000 depicting personal information and pulmonary diagnostic information pertaining to a specific user in accordance with several embodiments. The results of the data analysis are presented to the patient via the mobile application, and they may also be presented instead of or in addition to another person of the patient's choice using any suitable methods including visually, audibly, and/or tactile methods. In one example, an email can be sent to the patient's clinician with a report of the recording. Block 1002 of the user interface 1000 shows the patient's name and contact information. Block 1004 of the user interface 1000 shows exemplary results from an analysis of the user's pulmonary health data. Block 1006 shows an exemplary option for the user to view and compare past recordings and block 1008 shows an exemplary option allowing a third party such as the user's physician to manually review by viewing and listening to each recording.

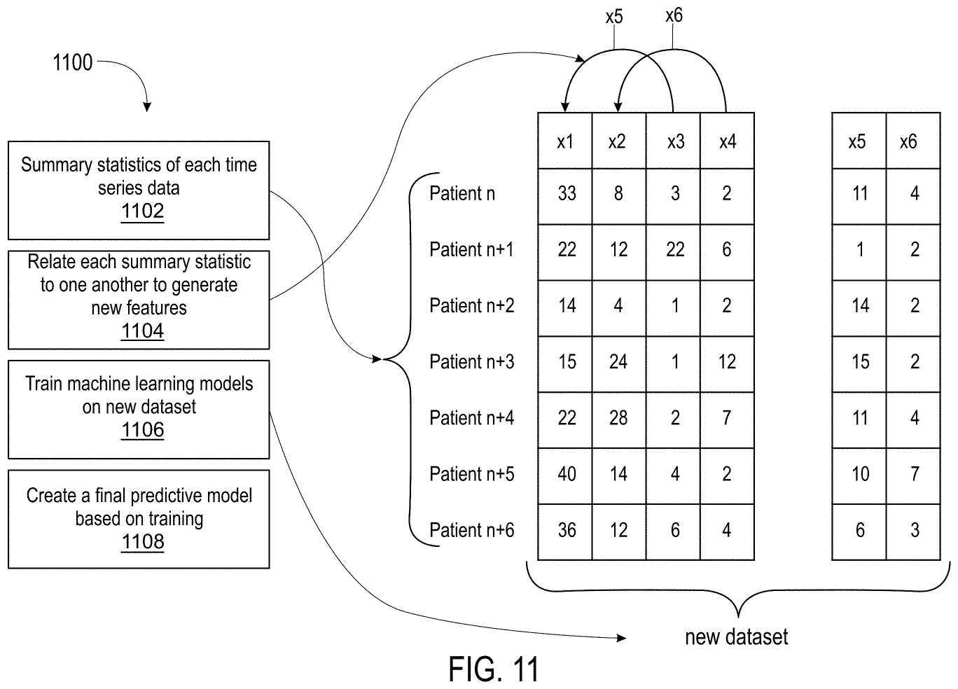

FIG. 11 illustrates a flow diagram 1100 of using data for model training in accordance with embodiments of the claimed subject matter. Step 1102 shows the use of summary statistics of each time series data pertaining to a plurality of the patients. Step 1104 shows the use of multiple summary statistics with comparison algorithms to generate new features. Step 1106 shows the use of the new datasets to train new machine learning models and step 1108 shows the step of the creation of a final predictive model based on the training of the machine learning models. Various other combinations of methods may be used to train any number of models which can be used for data analysis.

Some embodiments include an example non-invasive pulmonary congestion assessment system for training a model used with a non-invasive sensor device to estimate a patient's pulmonary congestion condition more accurately without imaging-based measurements of the patient's pulmonary congestion condition. An imaging-based measurement may include but is not limited to, MRI, CT-scan, sonography, Chest X-ray, angiography, and PET-scans. The system has at least one imaging-based measurement device configured to measure lung fluid data from a plurality of patients, at least one non-invasive sensor device including a plurality of sensors configured to capture non-invasive pulmonary congestion data from the plurality of patients; a computing device comprising a memory storing a plurality of instructions and a processor to execute the plurality of instructions. The processor is configured to match the lung fluid data with the non-invasive pulmonary congestion data from each of the plurality of patients over at least one temporal window corresponding to a specific measurement time period when both the lung fluid data and the non-invasive pulmonary congestion data were measured simultaneously. The processor also generates features from at least one portion of the non-invasive pulmonary congestion data that fall within each of the at least one temporal window, the features being processor-generated manipulations of raw data of the non-invasive pulmonary congestion data. The processor trains a machine learning model to estimate lung fluid data based on the generated features as independent variables and the lung fluid data as a first of at least one dependent variable; and then stores the machine learning model.

Another embodiment is directed to a non-invasive pulmonary congestion assessment system for training a machine learning model to estimate lung fluid data based on a non-invasive sensor device. The system includes a first computing device connected to at least one imaging-based measurement device to obtain lung fluid data of a plurality of users. It also may include a second computing device to receive the lung fluid data and non-invasive pulmonary congestion data, wherein the second computing device comprises a memory to store a plurality of instructions and a processor to execute the plurality of instructions. The processor is configured to: separately match lung fluid data with the non-invasive pulmonary congestion data. For each gold standard data, the processor generates features from at least one portion of the non-invasive pulmonary congestion data that fall within each of at least one temporal window; train a classification model and/or a regression machine learning model based on the generated features; and store the machine learning model.

A different embodiment is directed to an example system for training a non-invasive sensor device based on lung fluid data. The system includes a first computing device connected to a measurement device to obtain lung fluid data of a plurality of users; and a second computing device to receive the lung fluid data and non-invasive pulmonary congestion data, wherein the second computing device comprises a memory to store a plurality of instructions and a processor to execute the plurality of instructions. The processor is configured to match lung fluid data with non-invasive pulmonary congestion data, train a neural network model by using the lung fluid data as a gold standard, and store the neural network model.

Another system is described for capturing cardiovascular health data for managing heart failure. This includes handheld electronic device (HED) including an HED processor configured to execute communication application including instructions to register a user over the communication application by receiving at least one credential from the user for providing access to the communication application; receive demographic data pertaining to the user; receive pulmonary congestion questionnaire data from the user; and transmit a final dataset by compiling the demographic data and the pulmonary congestion questionnaire data of the user. The system also includes a computing device including memory and a processor coupled with the memory to execute a plurality of instructions pertaining to the management of heart failure, classification models, and machine learning models to estimate pulmonary congestion; a wearable device communicatively coupled with the handheld electronic device (HED) over a network, the wearable device including a Photoplethysmography (PPG) sensor to measure volumetric variations of blood circulation; and a non-invasive sensor device having a shape adapted to secure a plurality of components within the non-invasive sensor device. The plurality of components includes a soundwave transducer configured to capture auscultation signals indicative of pulmonary congestion of a user; an Inertial Measurement Unit (IMU) sensor configured to capture seismic auscultation signals indicative of the pulmonary congestion of the user; and a microcontroller configured to process data received from the PPG sensor, the soundwave transducer, and the IMU sensor to obtain non-invasive sensor data. The computing device receives the non-invasive sensor data and the final dataset from the communication application, stores in the memory the plurality of instructions; and executes the plurality of instructions including receiving, in at least one temporal window, a representation of data from at least one of the following: the PPG sensor, the soundwave transducer, and the IMU sensor; detecting features from at least one portion of the received representations of data that fall within each of the at least one temporal window; and applying a trained machine learning model to the detected features. The trained classification model and the machine learning model are configured to estimate pulmonary congestion of the user and adjust at least one medication of the patient based on the estimations of the pulmonary congestion of the user.

Additionally or alternatively, the computing device records or instructs the plurality of patients, their health care providers, or their agents to input demographic data and health questionnaire data of the plurality of patients. Additionally or alternatively, the plurality of sensors of the at least one non-invasive sensor device comprises a soundwave transducer, a Photoplethysmography sensor, and an Inertial Measurement Unit sensor.

Additionally or alternatively, the processor generates features based on at least one of sound data by the soundwave transducer and movement data by the inertial measurement sensor of the environment while the non-invasive cardiac health data is being collected from the at least one non-invasive sensor device.

Additionally or alternatively, the at least one invasive measurement device can be a catheter, implanted pressure sensor, and/or an implanted micro-computer. Additionally or alternatively, a second computing device is connected to the at least one invasive measurement device.

Additionally or alternatively, a wearable device worn by the user can be used to obtain physiological data of the user and transmit to a handheld electronic device over a network.

Additionally or alternatively, the physiological data obtained by the wearable device during the collection of pulmonary congestion measurement data is included in the external data.

Additionally or alternatively, a second computing device can be connected to the at least one invasive measurement device.

Additionally or alternatively, the stored classification model and the machine learning model are transferred to the non-invasive sensor device.

Additionally or alternatively, the machine learning model and classification model estimate pulmonary congestion data of a new patient using the at least one non-invasive sensor device.

Additionally or alternatively, the predicted pulmonary congestion data of the new patient is utilized to adjust medical treatment or provide a medical treatment recommendation for the new patient.

Additionally or alternatively, the machine learning model is stored in a second computing device, and the at least one non-invasive sensor device physically connected to or integral with the second computing device.

Additionally or alternatively, the plurality of sensors of the least one non-invasive sensor device includes a soundwave transducer, a Photoplethysmography sensor, and an Inertial Measurement Unit sensor, and the non-invasive sensor device is connected to or integral with a handheld electronic device.

In an embodiment, the processor of the HED (112) prompts the user to log in with a username. In an embodiment, the processor displays a result of the calculation on a display screen of the computing device. In an embodiment, the circuit board is further connected to at least one Photoplethysmography (PPG) sensor for measuring blood flow patterns indicative of the pulmonary health of the user. According to an embodiment herein, the present system is accessed and utilized by two types of users. The first type of user includes a patient when the data is being collected or the patients want to monitor themselves. The second type of user includes a healthcare professional who logs in to the software application and places the HED on the patient for monitoring purposes.

According to an embodiment herein, the present disclosure further provides a handheld electronic device (HED) for assessing pulmonary health. The handheld electronic device (HED) includes at least two ECG electrodes; at least one sound transducer; at least one Inertial Measurement Unit (IMU) sensor; and a microcontroller. The ECG electrodes are placed on the outer surface of the HED The ECG electrodes are configured to capture the electrophysiological data of the user. The sound transducer captures soundwave signals indicative of the pulmonary health of the user. The Inertial Measurement Unit (IMU) sensor to capture seismic and gyroscope signals indicative of the pulmonary health of the user and orientation of the HED The microcontroller transmits pulmonary health data received from the ECG electrodes, the sound transducer, and the IMU sensor to at least one of the HED and a computing device. At least one of the HED and the computing device is configured to: receive, in one or more temporal windows, a representation of data from one or more of the following when positioned against the thoracic cavity of the user: the IMU sensor signals, the plurality of ECG electrodes signals, and the sound transducer signals; detect features from at least one or more portions of the received representations of data that fall within each of the one or more temporal windows; identify patterns in the detected features based on one or more of the following models: a classification model and a regression model; and using the identified patterns, calculate a probability of whether the identified patterns correspond to a problem with the pulmonary health of the user and/or estimate a progression of a pulmonary health condition.

In an embodiment, the HED includes two or more IMU sensors, two or more sound transducers, and two or more PPG sensors. In an embodiment, the pulmonary health condition comprises lung fluid levels. In an embodiment, the handheld electronic device (HED) includes at least one Photoplethysmography (PPG) sensor to measure blood flow patterns indicative of the pulmonary health of the user.

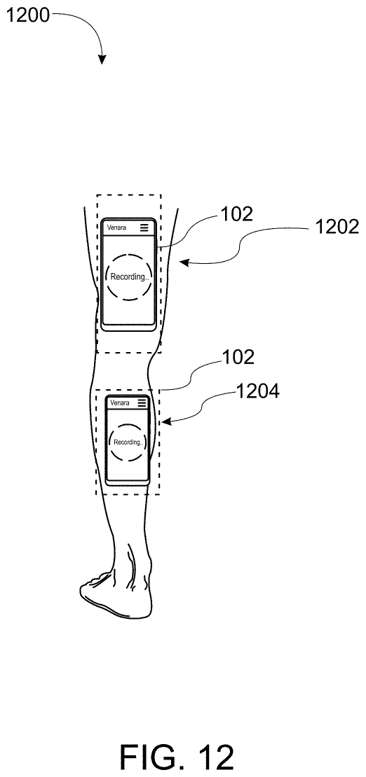

In an embodiment, casing 102 is placed in a lower limb region of the user to collect vein thromboembolism (VTE) risk data therefrom. FIG. 12 illustrates a perspective view 1200 of the placement of casing 102 on the lower limb of the patient in accordance with at least one embodiment of the claimed subject matter. The casing 102 is placed on the patient's lower limb to capture the VTE risk data. In some embodiments, the software application executed on the mobile phone may be used to direct the user to correct placement upon their thigh. Directions originating from the application may comprise a first step 1202 to place the casing 102 on the patient's thigh, and then in second step 1204, placing the casing on the calf of the patient to capture the VTE risk data. The patient has to keep recording/capturing the VTE risk data until the application instructs to stop. Then the user can press a stop button displayed on the application to upload the VTE risk data to the server. In an embodiment, the computing device identifies unique physiological markers of the user comprising previously collected sensor data. In an embodiment, the data indicating a high probability of VTE triggers a message transmission to a healthcare professional. In an embodiment, the casing can be worn as a wearable device and/or patch by using an adhesive material or a strap to ensure that the casing has contact with the patient's body and can be attached to the patient's body for longer periods of time. According to an embodiment herein, casing 102 is used as a patch with the ability to adhere to the patient's body. The patch can be placed on the side of the leg as well. The patch can be placed on the thoracic cavity of the patient. Further, the patch can be configured so that an ECG measurement can be taken multiple times over a period of time longer than 30 minutes.

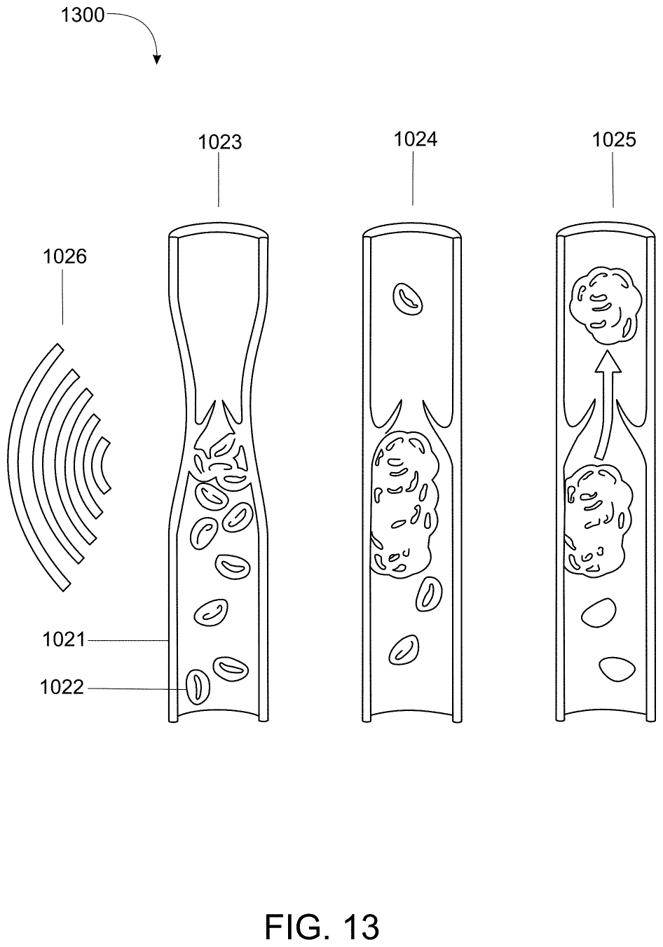

FIG. 13 illustrates a perspective view 1300 of the deep vein thrombosis (part of vein thromboembolism (VTE)) in accordance with at least one embodiment of the claimed subject matter. FIG. 13 depicts a representation of VTE by way of one deep vein thrombosis (DVT) example and one pulmonary embolism example and how these relate to signals that at least one IMU sensor may identify. As blood flow 1022 within a vein 1021 becomes clotted, thrombus starts building up and the beginning of DVT may be possible to observe 1023. This process of DVT continues and the thrombus continues to clot and occlude blood flow within the vein 1024. At some point said thrombus may become too big and be released in the rest of the cardiac system, known as an embolus 1025. This may result in pulmonary embolism and is potentially fatal. These internal venous dynamics provide signals 1026 that can be identified using a number of sensors, including but not limited to IMU sensors and acoustic sensors.

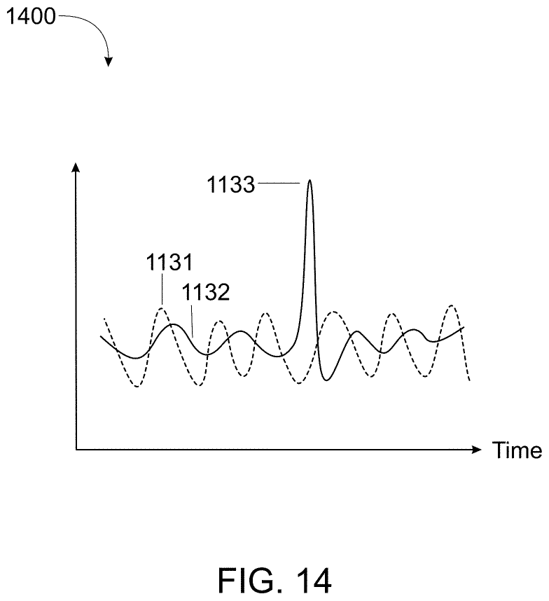

FIG. 14 illustrates a graphical representation 1400 of the IMU and/or microphone signal level in accordance with at least one embodiment of the claimed subject matter. FIG. 14 depicts a graph of a typical IMU data waveform. FIG. 14 is explained in conjunction with FIG. 13. Time is represented on the x-axis through units of tenths of a second. The y-axis is represented by IMU signals which may include but are not limited to units of specific force, angular rate, and orientation. When clots build-up and/or are released 1023, 1024, 1025, the internal dynamics of the vein change, and a waveform typically associated with smooth blood flow may temporarily be disrupted 1133. Normal blood flow can be expected to be represented by a more steady and recurring wavelike form 1131, 1132. Said disruptions may not be easily identifiable and require machine learning methods to detect microscopic perturbations and variance in venous blood flow. Such intervals may reveal underlying hemodynamic problems, thus representing another reason why unique data points relating to IMU and/or other sensor technology through the extended wear IMU and physiological sensor monitor described herein can be important for understanding the general health of a patient. The long-term data acquisition of these IMU data points, obtained through extended wear of the wearable monitor, can give the patient valuable insights into the patient's hemodynamic function and general physical health.

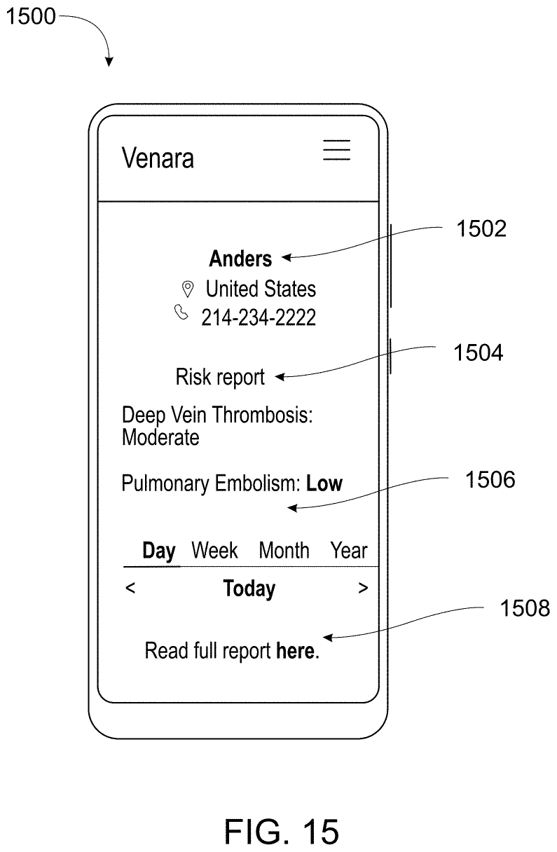

FIG. 15 illustrates a user interface 1500 to depict personal information and VTE diagnostic information pertaining to the user in accordance with at least one embodiment of the claimed subject matter. The results of the data analysis are presented through the mobile application to the patient and/or in any other way to a person of the patient's choice. For example, an email might be sent to the patient's clinician with a report of the recording. At block 1502, the user interface 1500 depicts patient names and contact information. At block 1504, the user interface 1500 depicts the results from the analysis of the VTE risk data. At block 1506, the user interface 1500 depicts that the user can view and compare past recordings. At block 1508, the user interface 1500 depicts that the physician can manually see and listen to each recording.

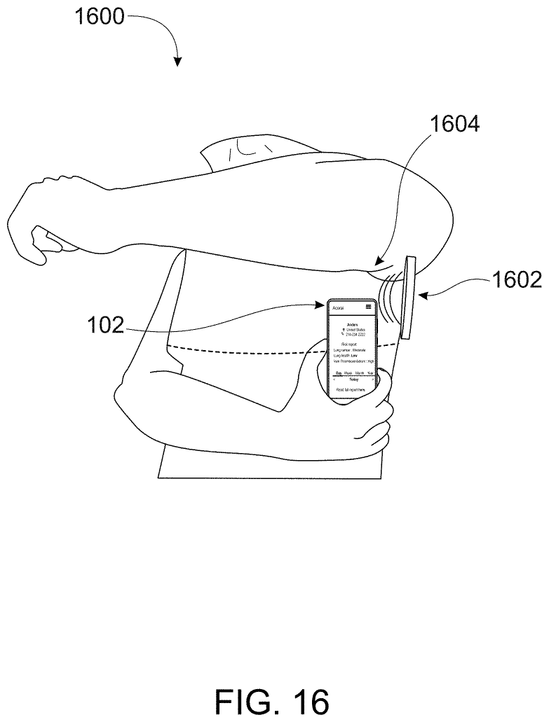

FIG. 16 illustrates an implementation 1600 of the present system with a sound transducer 1602 configured to emit soundwaves in the thoracic region of the user in accordance with at least one embodiment of the claimed subject matter. In some embodiments, the sound transducer 1602 emits soundwaves 1604 may be used in conjunction with the HED 112. The sound transducer 1602 may be any suitable sound transducer known to those skilled in the art or it may include more than one sound transducer working independently or together. The sound transducer 1602 may be attached to the HED 112 or it may be used as an external sound transducer 1602 with HED 112 with any suitable communication means between the sound transducer 1602 and the HED 112 such as a wired or wireless connection. In these embodiments, the sound transducer 1602 can be used to emit a noise in the body of the user that can be reflected and analyzed with one or more sensors such as internal or external sound transducers used with the HED 112. The resulting reflected noise or noises can be used by the embodiments to provide insights into obstruction, abnormalities, and pulmonary health of the user including the health of the user's lungs. In some embodiments, using the relative deviation from a normal and/or healthy lung baseline can be used. In an embodiment, the sound transducer 1602 is placed on the user's back to emit soundwaves into the thoracic region of the user whilst another sound transducer is simultaneously collecting data from another location of the thoracic region. Deviations in the recorded soundwaves from the expected soundwaves can be an indication of a pulmonary health condition, including but not limited to pulmonary congestion. In many of these embodiments, while recording using the HED 112 as described herein, the user may be prompted by the pulmonary monitoring application to make a low, continuous, and/or droning sound which may aid in that user's pulmonary health assessment through many mechanisms, including but limited to the increased vibrations originating from the thoracic region providing more pulmonary data to analyze and creating a baseline value of noise that other fluctuations in data can be compared to. In an embodiment, the implementation 1600 may comprise a transducer configured to emit vibrations into the body of the patient. In such an embodiment, the sound transducer 1602 may be replaced and/or additionally used.

Unless otherwise defined, all terms (including technical and scientific terms) used in this disclosure have the same meaning as commonly understood by one of ordinary skill in the art to which this disclosure belongs. It is to be understood that the phrases or terms used with the present inventive subject matter are for the purpose of description and not of limitation. As will be appreciated by one of the skills in the art, the present disclosure may be embodied as a device, system, and method, or computer program product. Further, the embodiments may take the form of a computer program product on a computer-readable storage medium having computer-usable program code embodied in the medium. The present systems, devices, and methods have been described above with reference to specific examples, however, other embodiments and examples than the above description are equally possible within the scope of the claimed subject matter. The scope of the disclosure may only be limited by the appended patent claims. Even though modifications and changes may be suggested by the persons skilled in the art, it is the intention of the inventors and applicants to embody within the patent warranted heron all the changes and modifications as reasonably and properly come within the scope of the contribution the inventors and applicants to the art. The scope of the embodiments of the inventive subject matter is ascertained with the claims as submitted at the time of filing the complete specification.

Regarding additional interpretation and construction of terms and steps herein, method steps are not in any specified order unless dictated by the context or specific wording. In addition , the use of a word in the singular form should be interpreted where the context allows or does not restrict so as to enable plurality or an “at least one” construction . Positional and directional terms described in this specification may be understood to be different than shown or described and should not limit the variations of embodiments possible from the claimed features that a person of ordinary skill in the art would understand from the specification, figures and claims. The term “and/or” in a list means all list items present , some list items present , or one of the list items present, unless such construction is limited by the context.

What is claimed is:

1. A system for assessing pulmonary health, the system comprising:
a handheld electronic device (HED) including a display screen, a processor, and a software application;
a casing comprising:
a plurality of ECG electrodes placed on an outer surface of the casing; and
at least one microcontroller;
at least one sound transducer configured to capture pulmonary signals indicative of pulmonary health;
at least one diaphragm to enhance pulmonary audio signals captured by the at least one sound transducer;
at least one Inertial Measurement Unit (IMU) sensor configured to capture seismic and gyroscope signals indicative of the pulmonary health of a user and an orientation of the casing;
at least one circuit board disposed within the casing and electrically connected with the plurality of ECG electrodes, the at least one microcontroller, the at least one sound transducer and the at least one IMU sensor,
wherein
the microcontroller is configured to transmit data received from the plurality of ECG electrodes, the sound transducer, and the IMU sensor to at least one of the HED and a computing device, wherein at least one of the HED and a computing device is configured to:
receive, in one or more temporal windows, a representation of data from one or more of the following when the casing is positioned against the thoracic cavity of the user: signals from the at least one IMU sensor, signals from the plurality of ECG electrodes signals, and signals from the at least one sound transducer;
detect features from at least one portion of the received representations of data that fall within each of the one or more temporal windows;
identify patterns in the detected features based on at least one of a classification model and a regression model; and
using the identified patterns, calculate at least one of a probability of whether the identified patterns correspond to clinically relevant indicators of a pulmonary health condition of the user and an estimate a progression of the pulmonary health condition.

2. The system according to claim 1, further comprising a second electronic device wearable by the user, wirelessly connected with the HED, wherein the second electronic device comprises:
one or more sensors to collect health data from the user; and
a wireless transceiver configured to establish a communication between the second electronic device and the computing device to transmit health data therebetween, wherein the computing device is configured to:
detect, based on the classification model, the presence of the indicators of a pulmonary health condition, and
estimate, using the indicators of the pulmonary health condition, based on the regression model, a severity of the pulmonary health condition.

3. The system according to claim 1, wherein the system further comprises an additional sound transducer configured to emit soundwaves into the thoracic region of the user.

4. The system according to claim 1, wherein the processor is configured to present to the user one or more commands to determine the positioning of the casing on the user's body.

5. The system according to claim 1, wherein the classification model is trained to classify an unhealthy lung caused by pulmonary congestion.

6. The system according to claim 1, wherein the regression model is trained to estimate a lung fluid level.

7. The system according to claim 1, wherein the classification model is trained based on data received from one or more of the following: lung computerized tomography (CT) scans, chest X-rays, spirometer data, magnetic resonance imaging (MRI) data, and sound transducer data.

8. The system according to claim 1, wherein the HED further comprises a battery configured to supply electrical power to the circuit board.

9. The system according to claim 2, wherein data indicating a high severity of a pulmonary health condition triggers a message transmission to a healthcare professional.

10. The system according to claim 1, wherein the processor is configured to display a result of the calculation on a display screen of a computing device.

11. The system according to claim 1, wherein the casing has a shape adapted to secure the HED to the casing or integrated with the HED

12. The system according to claim 1, the system further comprising at least one sound transducer configured to emit soundwaves into the thoracic region of a user.

13. A method for assessing pulmonary health with a casing connected to a handheld electronic device (HED), the casing including
a plurality of ECG electrodes placed on an outer surface of a casing, at least one diaphragm, at least one microcontroller, at least one sound transducer, and at least one Inertial Measurement Unit (IMU) sensor, and at least one circuit board, wherein the circuit board is connected to one or more of the ECG electrodes,
the method comprising:
positioning the casing against the thoracic cavity of a user;
capturing electrophysiological data of the user through the plurality of ECG electrodes;
capturing pulmonary signals indicative of the pulmonary health through the at least one sound transducer; and
capturing seismic and gyroscope signals indicative of the pulmonary health of the user and the orientation of the casing through the at least one Inertial Measurement Unit (IMU) sensor;
transmitting with the microcontroller pulmonary health data captured by the plurality of ECG electrodes, the at least one sound transducer, and the IMU sensor to at least one of the HED and a computing device;
receiving, in one or more temporal windows, by at least one of the HED and a computing device a representation of data from one or more of sensor signals from the at least one IMU sensor, signals from the plurality of ECG electrodes, and signals from the sound transducer;
detecting by at least one of the HED and a computing device features from at least one portion of the received representations of the data that fall within each of the one or more temporal windows;
identifying by at least one of the HED and a computing device patterns in the detected features based on one or more of a classification model and a regression model; and
using the identified patterns, calculating by at least one of the HED and a computing device a probability of whether the identified patterns correspond to clinically relevant indicators of a pulmonary health condition of the user and estimate a progression of the pulmonary health condition.

14. The method according to claim 13, further comprising the step of emitting, by an additional sound transducer, soundwaves into the body of the user.

15. The method according to claim 13, further comprising sensing temperature of the user's chest skin temperature by a temperature sensor in the casing.

16. The method according to claim 13, further comprising a step of presenting one or more commands to the user to inform the user of correct positioning of the casing on the user's body.

17. The method according to claim 13, further comprising a step of training a classification model to classify an unhealthy lung caused by pulmonary congestion.

18. The method according to claim 13, further comprising a step of training the regression model to estimate a lung fluid level.

19. The method according to claim 13, further comprising training the classification model based on data received from one or more of lung computerized tomography (CT) scans, chest X-rays, spirometer data, magnetic resonance imaging (MRI) data, and sound transducer data.

20. The method according to claim 13, further comprising a step of electrically supplying the casing power from a battery of the HED

21. The method according to claim 13, the step of calculating calculates the data indicating a high severity of a pulmonary health condition and a step of triggering transmits a message to a healthcare professional.

22. The method according to claim 13, further comprising a step of displaying on a screen of the HED a result of the step of calculating.
